(19) United States
(12) Patent Application Publication
(10) Pub. No.: US 2008/0282580 A1
(43) Pub. Date: Nov. 20, 2008

(54) Method and Apparatus for Curing Body Status

(76) Inventor: Kim Ji-Woog, Busan (KR)

Correspondence Address: IPLA P.A., 3580 WILSHIRE BLVD., 17TH FLOOR, LOS ANGELES, CA, 90010, US

(21) Appl. No.: 11/571,819

(22) PCT Filed: Jul. 11, 2005
(86) PCT No.: PCT/KR2005/002224

§ 371 Date: Jan. 8, 2007

(30) Foreign Application Priority Data
Jul. 10, 2004 (KR) 10-2004-0053818
Jul. 12, 2004 (KR) 10-2004-0053823
Jul. 12, 2004 (KR) 10-2004-0054145
Jul. 14, 2004 (KR) 10-2004-0054594
Jul. 14, 2004 (KR) 10-2004-0054600
Jul. 14, 2004 (KR) 10-2004-0054605
Jul. 14, 2004 (KR) 10-2004-0054606
Jul. 14, 2004 (KR) 10-2004-0054615
Jul. 16, 2004 (KR) 10-2004-0055395
Jul. 28, 2004 (KR) 10-2004-0059438
Jul. 29, 2004 (KR) 10-2004-0059457
Aug. 2, 2004 (KR) 10-2004-0060932
Aug. 4, 2004 (KR) 10-2004-0061258

(51) Int. Cl. A61F 5/14

(52) U.S. Cl. 36/140

(57) ABSTRACT

The present invention is an apparatus for learning and leading a user to have a posture according to a corresponding purpose at his/her foot, and can cure and improve a state of a target human body according to the corresponding purpose. The apparatus for curing a human body using a pressure under foot, is characterized in that a protrusion part and a sinking part are provided under the foot.

### TECHNICAL FIELD

The present invention relates to an apparatus for curing a human body using a pressure under feet.

### BACKGROUND ART

In a conventional art, there have been some slipsoles where its specific portion is protruded to stimulate a specific nerve or acupuncture spot for the purpose of acupressure and reflex zone therapy, and some inventions where a specific portion is protruded from a surface of the slipsole for the purpose of walking or a specific posture. However, they do not have sufficient understanding for relation of foot and compensation, and do not consider characteristics of various organs, an effect of curing could not be expected. Human body has its own inherent property. The human bodies are not the same in state. Here, a deformity of a musculoskeletal system such as genu varum, genu valgum, flatfoot, pes cavus, and hallux valgus is included. In case where the human body is an object, this should be carefully considered. However, in a conventional invention, they are not considered, and since there is only a specific type of a model, when application is actually made to various states of the human body, it is rather greatly worried that compensation distorted into an unexpected third type is requested or a problem itself results in serious lesion and deformity.

### DISCLOSURE OF INVENTION

### Technical Problem

Accordingly, the present invention is greatly distinguished from a conventional art in its object, and provides an apparatus being characterized by learning and leading a user to support his/her weight at his/her feet according to a corresponding purpose, curing the feet and a posture, and further curing and remedying a physical figure, a constitution, a lesion, and a deformity, and being characterized by being capable of being positively utilized in improving a state of a human body such as athletic power, walking, and beauty. The present invention is characterized by providing an effective means applied to various human bodies and positively receiving his/her request and curing the human body in a state conforming to his/her request in case where there is an ideal state of the human body, a state of the human body conforming to a curing purpose, or a specific state of the human body requested by a user in application, and by differentiating a construction according to a corresponding purpose and being flexibly applied to various human bodies.

### Technical Solution

To achieve these and other advantages and in accordance with the purpose of the present invention, as embodied and broadly described, there is provided an apparatus for curing a human body using a pressure under foot, in which a protrusion part and a sinking part are provided under the foot.

The apparatus is a slipsole; the slipsole comprises a top plate, a middle plate provided at a lower side of the top plate and having at least one hole, a bottom plate provided at a lower side of the middle plate, and an insertion member inserted into the hole of the middle plate; and the protrusion part or the sinking part may be formed by a difference between the insertion member and the middle plate. At this time, the insertion member is named as a protruding unit or a sinking unit according to its usage above and below.

The apparatus may additionally include an electronic curing device, and the electronic curing device includes a foot bottom pressure sensor positioned on a foot bottom surface or a lower surface of an object corresponding to the foot bottom surface, and sensing a pressure by parts where weight is loaded; a signaling unit for generating a signal; and a curing program master for receiving a curing program having information on a position and a degree by parts where the weight should be loaded and its corresponding information on an operation content of the signaling unit and stored in a memory, and receiving data sensed by the foot bottom pressure sensor, and comparing and analyzing the received information and information of the curing program, and controlling the signaling unit according to the information on the operation content of the signaling unit included in the curing program.

In application of the present invention, a reaction to the sinking part can depend on a person. Accordingly, the protrusion part and the sinking part can be differently applied, depending on the person, in consideration of user's property and reaction.

For one example, the present invention can be used for curing a state of a center of gravity of human body. This can be achieved by learning and leading the user so that the weight loaded on the corresponding structure can be controlled in degree. In case where the protrusion part and the sinking part are positioned at a position selected for the purpose of curing according to need, and the weight is less loaded, it can be expected that the center of gravity of the human body rises, and in case where the weight is more loaded, it can be expected that the center of gravity of the human body falls. This is in the same context as that of a principle that the center of gravity of the human body rises when the person walks on a thin ice, and the center of gravity of the human body falls when the person walks on a spongy object.

In application, for one example, in case where the protrusion part and the sinking part are large-sized and result in pain, the user would avoid the pain. Therefore, it can be expected that the center of gravity of the human body rises. On contrary, in case where the protrusion part and the sinking part are small-sized and soft and the user is learned and lead to allow the weight to be loaded on the protrusion part and the sinking part, the user can act and accurately recognize them. Therefore, it can be expected that the center of gravity of the human body falls. However, this should be carefully selected in consideration of a property and a reaction of a corresponding organic body. For one example, in case where the a large sized and high elastic protrusion part is applied, the person positively reacting to the protrusion part may load much weight to sufficiently step on the corresponding protrusion part. On contrary, the person having opposition to the protrusion part may load less weight on the corresponding protrusion part so as to avoid the protrusion part. Even the sinking part can be understood in the same context.

In application of the present invention, the positions of the protrusion part and the sinking part may not be consistent with the ideal positions of the corresponding organic body at which the weight should be supported. This is because a state of a musculoskeletal system such as genu varum, genu valgum, flatfoot, pes cavus, and hallux valgus is considered in the corresponding organic body. For one example, in case where the weight is biased and built at an outer side of the foot and there are deformities of the flatfoot and the genu varum, when the weight is simply supported with a standard of the ideal position at which the weight should be supported in the corresponding organic body, an actual effect of curing cannot be expected and in addition to this, there is a concern that the deformities rather get worse as time lapses.

For one example, in case where a state where the weight is biased at a specific position is repeated and maintained for a long time, thereby resulting in the deformity of the musculoskeletal system including the foot, in curing, the protrusion part or the sinking part or they all are placed at an opposite side corresponding to the position on the foot where the weight is biased due to a corresponding variation so that with a standard of portions corresponding to them, the weight can be supported. This can be frequently utilized in a curing process.

For one example, it can be positively utilized for curing a scoliosis. When the weight is laterally biased, the scoliosis is caused. In case where the state where the weight is biased to a specific foot is repeated and maintained for a long time, thereby constructing the scoliosis, a state where the weight is supported at both feet can be contrary methodically for the curing effect in the curing process. Basic solution is to evenly support the weight at both feet, but this is a method being capable of being time-limitedly used in consideration of the curing effect.

In general, in case where the weight is biased to the specific foot, the foot where the weight is biased is in trend of relatively supporting the weight with a standard of rear and outer sides of the foot bottom surface, and the other foot is in trend of supporting the weight with a standard of front and outer sides or rear and inner sides (a relative position, not an absolute position, on the foot) of the foot bottom surface. In case where the user having the scoliosis resulting from the weight biased and built at the left foot is cured on the assumption of this trend, in the slipsole corresponding to the right foot, the protrusion part or the sinking part or a combination thereof is more placed at a portion corresponding to a rear and inner side of the foot bottom surface, and in the slipsole corresponding to the left foot, the protrusion part or the sinking part or a combination thereof is less placed at a portion corresponding to a front and inner side of the foot bottom surface so that the weight can be supported with a standard of the portions of the feet corresponding to them. Here, a difference in size between the protrusion part and the sinking part is to allow the user to more easily recognize a larger-sized part thereby leading such that the weight can be loaded or biased more frequently and for a long time, and leading such that more weight can be relatively loaded with a standard of the larger-sized part. However, the person's reaction to the protrusion part can be different depending on the person and therefore, it does not correspond to all cases. In case where the person reacts contrarily, it is possible to contrarily apply the protrusion part and the sinking part.

In another method, in case where at a portion where the weight is more biased, the protrusion part is placed to be small-sized and slightly sharp to a degree of properly causing the pain, and at a portion where the weight is less biased, the protrusion part is placed to be large-sized and wide so that the weight can be supported with a standard of the protrusion part, the user can react and avoid the pain of the foot bottom surface and therefore, it can be expected that the weight can be supported on the foot to the contrary of a state of that time.

In addition, the protrusion part or the sinking part or the combination thereof is placed only at a rear and inner side of the slipsole corresponding to the right foot, and with a standard of this, the weight can be supported. In general, the person having the scoliosis inclines to have a large pace about the foot in a direction where the weight is biased to and built in its walking. Accordingly, in that case, a heel can be lately set apart from the ground when the user walks for the purpose of recognizing the protrusion part and the sinking part. In that case, it can be expected to increase the pace about the right foot.

In the above example, in case where the user's musculoskeletal system is adapted, it can be expected to cure the scoliosis.

The above application example is one example and does not applied to all cases. In addition, according to the corresponding purpose, various methods can be employed. The protrusion part and the sinking part can be varied in position, number, type, size, material, and construction according to need of the corresponding purpose.

In application of the present invention according to the corresponding purpose, a result of applying the corresponding purpose to the human body can be a little or a lot different from the state of that time. In application of the corresponding purpose, a degree of application can be controlled or progressed in a gradual or sudden fashion. In case where the curing is performed in a sudden fashion, a burden can be laid on the musculoskeletal system, thereby rather resulting in lesion and deformity. However, this can be different depending on case, and the contrary to this can be effective in curing according to case. For one example, in case where the curing is not easy in the built state of that time, it is required to produce a contrary environment to this, and secure flexibility in the musculoskeletal system.

In application, the present invention follows “Ground surface and support structure”, and registration writings in Korean copyright deliberation and conciliation committee, written by Ji Uk KIM and entitled “ground surface and support structure” having the registration number of C-2004-002860”. The technological spirit is used as a theoretical basis useful in analyzing, evaluating and improving the state of the human body being an object. However, as well known, the human body is not yet made clear in parts more than parts made clear. Accordingly, the technological spirit can be modified and added in the future. In case where there is a new theory for criticizing or substituting for the technological spirit, the technological spirit can refer or follow the new theory in application of the present invention.

Main Technological Spirit of the Present Invention

<Ground Surface and Support Structure>

1-1. Evaluation of Compensatory Mechanism

The compensatory mechanism built for a long time has optimized muscle and skeleton, and an exercise characteristic optimized to them. In case where there is a variable, its corresponding posture can be compensated relatively in real time. However, in the formed posture, the optimization of the muscle is not performed. This is because a predetermined time is taken to optimize the muscle to the corresponding posture. Therefore, the exercise characteristic optimized to the corresponding posture is not reflected. Accordingly, the evaluation and analysis of the corresponding organic body is required to be based on a state where the variable applied to the corresponding organic body and its compensation are built for a relatively long time. At this time, the inventive technological spirit can be used as a useful means in evaluation and analysis.

1-2. Composition and Decomposition of Force

The posture is naturally formed through a relation of the ground surface and the support structure. The variation of the support structure is a variable of a segment. In the technological spirit, the variables requiring the compensation are independently analyzed if possible. Further, a description of the relation of the variable and the compensation is limited that only one variable analyzed in this research singly is applied to the human body being the object. This is to avoid a duplicate description and help understanding. However, actually, the human body is in trend of having a complicated relation of two or more variables analyzed in this research. Each variable requires the compensation corresponding to the corresponding variable. The compensation required by each variable is similar with, corresponds to, or contrary to those required by other variables. In case where the variables requiring the similar, corresponding or contrary compensation are concurrently related to the human body, the compensation result is of a type where the compensations requiring the variables are negotiated and compromised. At this time, “composition and decomposition of force” can be understood through a physical law.

The variables independently analyzed in this research can be understood in the same context as a concept of three dimensions expressing a dimensional object through a combination of X, Y and Z axes each having a value. Here, there is provided a method in which the analyzed variables make it possible to decompose and analyze the related variables in evaluating the compensatory mechanism formed by complicatedly relating two or more variables, and facilitate to again combine and evaluate the variables. In addition, there is provided a method of facilitating to understand a problem of the corresponding compensatory mechanism and provide and apply a new variable being capable of obtaining the curing effect, in case where the curing is needed. In case where the variable exists, its corresponding compensation is performed. This can be defined in such a manner that as the variable is relatively large in magnitude, its corresponding compensation is large. In case where the compensation corresponding to the variable is not appropriately performed, this can result in the lesion and the deformity.

1-3 Exception of Compensation

In the technological spirit, the variable of the segment is analyzed variously. These variables are selected in conformity with personalities of the corresponding organic bodies, respectively. In various compensatory mechanisms, the personality of the corresponding organic body is the most important variable. In this case, a cause of a feature of the corresponding organic body such as a personality, a cause of the state of the human body, an environmental main cause required by this, and the state of the human body required by this can be different. In that case, they can be causes of the lesion and the deformity. The person's posture is naturally formed through the relation of the ground surface and the support structure. This human body state can be described through the physical law using a human body's reaction against the gravity. However, the person's posture not always reacts to the physical law optimally. The compensation for the segment not following the physical law is mostly caused by the environmental main cause. As an easy example, there is exemplified a segment variable resulting from a beauty desire or a segment variable where a political purpose executed in relation with a third party is reflected.

2. Evaluation of Spinal Curvature

The spinal curvature can be expressed in various languages. This is different depending on an academic circle and a scholar. This makes it difficult to describe and exchange. Accordingly, a predefined term is required. In a sagittal plane, a lumbar spine is protruded to the front, and a thoracic spine is protruded to the rear. In this technological spirit, they are defined as lumbar lordosis and thoracic kyphosis, respectively. Besides, in the scoliosis, the technological spirit defines a left convexly protruded part and a right convexly protruded part as a left protrusion and a right protrusion, respectively. This can be fragmented and expressed in each lumbar spine and thoracic spine. For one example, expression of “lumbar spine is left protruded and thoracic spine is right protruded” is provided.

Spine is comprised of cervical spine, thoracic spine, lumbar spine, sacral spine, and coccygeal spine. They are connected organically. Accordingly, their variations have influence with one another. In the technological spirit, the variations are variously analyzed. The variation requests the spine for its corresponding compensation, respectively. However, in case where they are concurrently related with the spine, they make the evaluation difficult more or less. For one example, in case where a hip joint rotates internally, the lumbar lordosis increases and the thoracic kyphosis decreases. Further, in case where the weight is biased at an inner side of the foot, the lumbar lordosis increases and the thoracic kyphosis decreases. In case where they are concurrently related, the lumbar lordosis will more increase and the thoracic kyphosis will more decrease. The position of the foot where the weight is leadingly loaded incline to be relatively consistent with a gravity center of a head on a vertical plane in the sagittal plate (This corresponds to all compensatory mechanisms. At this time, the corresponding human body is naturally in a standing state). Specifically, this should be considered importantly in case where two or more variables are concurrently related.

The spine is connected organically. Accordingly, the variation of the head has influence on the related spine. It has influence on the cervical spine and an upper part of the thoracic spine. The two variables request the head for protraction. Accordingly, as a result of compensating the two variables, the protraction of the head is large. In a result of varying upper parts of the cervical spine and thoracic spine together with the protraction of the head, the corresponding state can be thought as if kyphosis increases at an upper portion of the thoracic spine. In a case, this state is evaluated as the lumbar lordosis and the thoracic kyphosis all greatly increasing. However, this is not desirable. In accurate evaluation of this, it is desirable to understand that the lumbar lordosis greatly increases and the thoracic kyphosis greatly decreases, and the head is greatly protracted, thereby causing the front variations of the upper portions of the cervical spine and the thoracic spine having influence on this. This is a useful method being capable of reducing a disorder in understanding a correlation of the variable and the compensation. If the variables are distinguished and experimented, this is well proved. Accordingly, in case where several variables are related complicatedly, it is desirable that the evaluation of the spinal curvature follows the analysis of this research, and the variation of the head is appropriately considered and understood. In this technological spirit, the cervical spine is not considered relatively. The cervical spine is properly adapted to the state of the associated segment.

4. Bias and Compensation of Weight

The segment is connected organically. The variable of the specific segment has influence on other segments. In case where the weight is biased in front of the foot, it is inclined to accompany an outer bias of the weight on the foot and foot abduction. In case where the weight is biased in rear of the foot, it is inclined to accompany an inner bias of the weight on the foot and foot adduction. In case where the weight is biased at an inner side of the foot, it is inclined to accompany a rear bias of the weight on the foot and the foot adduction. In case where the weight is biased at an outer side of the foot, it is inclined to accompany a front bias of the weight on the foot and the foot abduction. Together with this, in case where the weight is biased to the foot, a muscular stress of a lower limb gets unbalanced. The unbalance of the muscular stress of the lower limb becomes the variables of hip joint inner and outer rotation. In case where the weight is biased at front and outer sides of the foot, it is inclined to accompany the outer rotation of the hip joint. In case where the weight is biased at rear and inner sides of the foot, it is inclined to accompany the inner rotation of the hip joint. In case where they are accompanied, all or some can be accompanied.

4-1. Compensation in Case where Weight is Biased at a Front Side of Foot

The hip joint is extended and externally rotated, and a pelvis is front-varied and posterior tilted. The lumbar lordosis decreases and the thoracic kyphosis increases. A scapula is extended. The center of gravity of the human body inclines to fall. In such a case, the scapula falls. The head inclines for retrusion and forward flexion. The forward flexion inclines to be great. A knee joint inclines to be curved. A longitudinal arch reduces. The toe is extended. In case of being natural, the toes are widened therebetween. In case where it is built for a long time, hammertoes and genu varum are obtained.

4-2. Compensation in Case where Weight is Biased at a Rear Side of Foot

The hip joint is curved and internally rotated, and the pelvis is rear varied and anterior tilted. The lumbar lordosis increases and the thoracic kyphosis decreases. The scapula is curved. The center of gravity of the human body inclines to rise. In such a case, the scapula elevates.

The head inclines for protraction and backward flexion. The backward flexion inclines to be great. The knee joint is extended. The longitudinal arch increases. The toe is curved. The toes are widened therebetween, and a great toe inclines to slightly curve toward a second toe. In case where it is built for a long time, claw toe, genu recurvatum, and genu valgum are obtained.

4-3. Compensation in Case where Weight is Biased at an Inner Side of Foot

The hip joint is curved and internally rotated, and the pelvis is rear varied and anterior tilted. The lumbar lordosis increases and the thoracic kyphosis decreases. The scapula is curved. The center of gravity of the human body inclines to rise. In such a case, the scapula elevates.

The head inclines for protraction and backward flexion. The knee joint is extended. A longitudinal arch increases. The toe is curved. The toes are widened therebetween, and a great toe curves toward a second toe. In case where it is built for a long time, genu recurvatum, and genu valgum are obtained.

4-4. Compensation in Case where Weight is Biased at an Outer Side of Foot

The hip joint is extended and externally rotated, and the pelvis is front varied and posterior tilted. The lumbar lordosis decreases and the thoracic kyphosis increases. The scapula is extended. The center of gravity of the human body inclines to fall. In such a case, the scapula falls. The head inclines for retrusion and forward flexion. The knee joint inclines to be curved. The longitudinal arch reduces. The toe is extended. In case of being natural, the toes are widened therebetween. In case where it is built for a long time, genu varum is obtained.

5-1. Adduction and Compensation of Foot

The hip joint is curved and internally rotated, and the pelvis is rear varied and anterior tilted. The lumbar lordosis increases and the thoracic kyphosis decreases. The scapula is curved. The center of gravity of the human body inclines to rise. In such a case, the scapula elevates. The head inclines for protraction and backward flexion.

5-2. Abduction and Compensation of Foot

The hip joint is extended and externally rotated, and the pelvis is front varied and posterior tilted. The lumbar lordosis decreases and the thoracic kyphosis increases. The scapula is extended. The center of gravity of the human body inclines to fall. In such a case, the scapula falls. The head inclines for retrusion and forward flexion.

6-1. Adduction and Compensation of Hip Joint

The hip joint is curved, and the pelvis is rear varied and anterior tilted. The lumbar lordosis increases and the thoracic kyphosis decreases. The scapula is curved. The center of gravity of the human body inclines to rise. In such a case, the scapula elevates. The head inclines for protraction and backward flexion.

6-2. Abduction and Compensation of Hip Joint

The hip joint is extended, and the pelvis is front varied and posterior tilted. The lumbar lordosis decreases and the thoracic kyphosis increases. The scapula is extended. The center of gravity of the human body inclines to fall. In such a case, the scapula falls. The head inclines for retrusion and forward flexion.

7-1. Pelvis Anterior Tilting and Muscle

A hip joint internal rotation muscle, a trunk flexion muscle, a hip joint flexion muscle, a knee joint extension muscle, an ankle joint dorsal flexion muscle, and a scapula flexion muscle are overstressed. In general, the center of gravity of the human body rises. In that case, a scapula elevator muscle and a thoracic respiration muscle are overstressed.

7-2. Pelvis Posterior Tilting and Muscle

A hip joint external rotation muscle, a trunk extension muscle, a hip joint extension muscle, a knee joint flexion muscle, an ankle joint bottom flexion muscle, and a scapula extension muscle are overstressed. In general, the center of gravity of the human body falls. In that case, the scapula elevator muscle is weakened in stress and the thoracic respiration muscle is overstressed.

8-1. Muscle in Case where Weight is Biased in Front of Foot

The hip joint external rotation muscle, the trunk extension muscle, the hip joint extension muscle, the knee joint flexion muscle, the ankle joint bottom flexion muscle, and the scapula extension muscle are overstressed. In general, the center of gravity of the human body falls. In that case, the scapula elevator muscle is weakened in stress and the thoracic respiration muscle is overstressed.

8-2. Muscle in Case where Weight is Biased in Rear of Foot

The hip joint internal rotation muscle, the trunk flexion muscle, the hip joint flexion muscle, the knee joint extension muscle, the ankle joint dorsal flexion muscle, and the scapula flexion muscle are overstressed. In general, the center of gravity of the human body rises. In that case, the scapula elevator muscle and the thoracic respiration muscle are overstressed.

8-3. Muscle in Case where Weight is Biased at Inner Side of Foot

The hip joint internal rotation muscle, the trunk flexion muscle, the hip joint flexion muscle, the knee joint extension muscle, the ankle joint dorsal flexion muscle, a lower-limb inner muscle and the scapula flexion muscle are overstressed. A lower-limb outer muscle is weakened in stress. In general, the center of gravity of the human body rises. In that case, the scapula elevator muscle and the thoracic respiration muscle are overstressed.

8-4. Muscle in Case where Weight is Biased at Outer Side of Foot

The hip joint external rotation muscle, the trunk extension muscle, the hip joint extension muscle, the knee joint flexion muscle, the ankle joint bottom flexion muscle, the lower-limb outer muscle and the scapula extension muscle are overstressed. The lower-limb inner muscle is weakened in stress. In general, the center of gravity of the human body falls. In that case, the scapula elevator muscle is weakened in stress and the thoracic respiration muscle is overstressed.

9. Scoliosis

In case where the weight is laterally biased, the weight is biased at an outer side of the foot. The person where the weight is frequently laterally biased inclines to repeat a left bias and a right bias of the weight with a period of a predetermined time rather than taking a right posture. Accordingly, in case where the lateral bias of the weight is repeated and maintained for a long time, the weight is biased and built at the outer sides of the feet. The person where the weight is frequently laterally biased inclines to relatively perform the bias of a specific direction for a longer time, rather than balance the lateral bias of the weight in both directions. In case where this is repeated and maintained for a long time, the human body is optimized and built in a state of that time. At this time, optimization is of a type built with a standard of a center of a position where the weight bias is relatively leadingly performed. This is a cause of the scoliosis. In case where the bias is repeated and maintained for a long time, the degree of the lesion and the deformity in the spine increases to some extents. In general, on the foot where the weight is biased, the weight is leadingly loaded centering on the rear and outer sides of the foot in comparison to the other foot.

9-1. Scoliosis in Case where Sagittal Axis of Foot and Sagittal Axis of Human Body are In Parallel

A tiptoe faces to the front. Hereinafter, only a case that the weight is biased to the left will be described. However, a contrary case can be also understood in the same context. In case where the weight is biased to the left foot, the pelvis is left varied. As the bias increases, the variation increases. In case where the weight is supported in majority by the left foot, the gravity center of the pelvis and the position of the left foot where the weight is leadingly loaded are relatively consistent on the vertical line.

The pelvis is right tilted on a frontal plane. The hip joint and the knee-joint of the left lower limb are hyper extended. The lumbar spine is right protruded, and the thoracic spine is left protruded. A shoulder girdle is left tilted, and the head is buckled. In case where there is an intentional try to erect the shoulder girdle and the head upright, the left scapula is elevated. However, even though there is the intentional try, the head inclines to show a continuous trend of buckling. In general, the scoliosis is smoothly curved over the entire spine. However, in case where there is the intentional try to erect the shoulder girdle and the head together at the scoliosis built for a long time, the intentional try is contrary to a natural compensation and therefore, the abrupt scoliosis is formed at a specific portion of the spine, thereby making irregular. The head is in trend of the buckling, the forward flexion, and the retrusion.

9-2. Scoliosis in Case where Sagittal Axis of Foot is Abducted at Sagittal Axis of Human Body

The foot is abducted at the sagittal axis of the human body, and eyes face to the front (f). Here, only a case where the weight is left biased will be described. However, a contrary case can be also understood in the same context. FIG. 12 illustrates the pelvis and the foot on the horizontal plane. FIG. 12(1) shows a state where the weight is not biased, and FIG. 12(2) shows a state where the weight is left biased. In case where the weight is biased to the left foot (a), the pelvis (b) is left varied and together, the left pelvis is rear rotated and the right pelvis is front rotated (2). As the weight bias is increased, the compensation is increased. In case where most of the weight is supported by the left foot, the center of the pelvis and the position of the foot where the weight is leading loaded are relatively consistent on the vertical plane, and a coronal axis of the pelvis and the sagittal axis of the foot are intersected, thereby forming an angle close to a right angle (2). On the frontal plane, the pelvis is right tilted. The hip joint and knee joint of the left lower limb are hyper extended. The thoracic spine is right protruded, and the lumbar spine is left protruded. The shoulder girdle is left tilted, and the head is buckled. An upper limb is naturally rotated to the left together with the rotation of the pelvis. However, the eyes face to the front (f) before the lateral bias of the weight. Accordingly, it is inclined to right rotate toward the front (f). Hereinafter, it is assumed that there is right rotation toward the front (f). The rotation is relatively evenly made over the entire human body. The upper segment of the foot is relatively gradually evenly rotated toward the front (f) with a standard of the left foot, and the head finally rotated so that rotation is finished. However, it is inclined not to rotate up to an initial position before variation. The left scapula is bent to incline to help a front surface of the upper limb to be closer toward the front (f). As a result of rotation, in the spine, the scoliosis is performed together with the rotation toward the front (f). In case where there is the intentional try to erect the shoulder girdle and the head upright, the left scapula is elevated. However, the head inclines to show a continuous trend of buckling.

In this case, a change of the spinal curvature is complex. In case where the weight is laterally biased, the weight is biased to an outer side of the foot. Further, it is inclined that the muscle of the lower limb is uneven so that the hip joint is internally and externally rotated (In many cases, the hip joint is relatively externally rotated). In addition, in case where the variables are all accompanied, it is inclined that the extension and external rotation of the hip joint is great so that the weight is biased to a front side of the foot. Accordingly, in this case, in the spinal curvature, the accompanying variable and its relation degree should be considered. The head inclines for buckling, forward flexion, retrusion, and right rotation.

10-1. Forward and Backward Flexions of Head, and Eye

If the head is in the forward flexion, eyebrows incline for elevation and abduction, and if the head is in the backward flexion, the eyebrows incline for falling and adduction.

10-2. Protraction and Retrusion of Head, and Eye

If the head is protracted, the eyebrows incline for falling and adduction, and if the head is retruded, the eyebrows incline for elevation and abduction.

10-3. Lateral Flexion of Head, and Eye

Hereinafter, only a case where the head is buckled will be described. However, a contrary case can be also understood in the same context. In a normal state, a virtual line linking both eyes is horizontal. In case where the head is buckled, the virtual line is left tilted. On the horizontal line, a left eye is positioned under a right eye. Accordingly, an object positioned at the same height of the eye or over the eye is clearly recognized by the right eye. In this case, it is inclined to elevate the left eyebrow, and tract a left eyelid. This can be presumed to result from the try to similarly secure eyesight of the both eyes at the height of the eye or over the eye. In case where this state is repeated and maintained for a long time, it is inclined that the right eye and the left eye become a main eye and a sub eye.

10-4. Lateral Rotation of Head, and Eye

Hereinafter, a description is based on “9-2. Scoliosis in case where sagittal axis of foot is abducted at sagittal axis of human body”. Accordingly, the variable and the compensation are progressed on the same extension line. In the “9-2. Scoliosis in case where sagittal axis of foot is abducted at sagittal axis of human body”, the head is right rotated toward the front (f) being an eye direction before the weight is left biased. In the result, the head inclines not to rotate up to the initial position. Hereinafter, such a thing is assumed. In this case, if the eyesight forwards to the front (f), the eyesight should face to the right. The object is relatively recognized through the right eye. In case where this state is repeated and maintained for a long time, it is inclined that the right eye becomes the main eye and the left eye becomes the sub eye. Together with this, the head inclines for buckling. In case where the head is buckled, a change of the eye resulting from the buckling of the head is concurrently performed.

11. Compensation at Nose

A structure of a nose is organically combined with an upper jaw tooth. Accordingly, a variation of the upper jaw tooth becomes a variation of the nose. The variation of the nose follows the variation of the upper jaw tooth. Relatively, the variation is greatly made under a septal cartilage structurally being in contact with the upper jaw tooth.

12-2. Variation of Tooth

Hereinafter, a front of the tooth is defined as an outer side, and its rear is defined as an inner side. In case where an intraoral pressure and a pressure of tongue are related with the tooth, this acts an inner pressure to the tooth. A stress and a weight of an obicularis oris muscle covering the tooth act as an outer pressure to the tooth. At a top, the upper jaw tooth acts as the outer pressure to the lower jaw tooth, and the lower jaw tooth acts as the inner pressure to the upper jaw tooth. A natural bite state is a state where the inner and outer pressures are balanced. This variation becomes the variation of the tooth. At this time, the variation of the tooth can be described through the physical law.

13. Variation of Lower Jaw

If a specific segment is varied, the human body is compensated at other segments and maintains a balance. However, in case where the variation is requested for the lower jaw in a process of natural compensation, the variation of the lower jaw is much different depending on the person. The compensation required for the lower jaw is properly reflected or not on the lower jaw. In addition, a distortion to a third type is also frequently generated. This results from a main cause such as the feature of the organic body and the environmental main cause. For one example, a state or a variation of the lower jaw resulting from an aesthetic desire, complement for complex, and a political purpose can be exemplified. The technological spirit is relatively for the general. Accordingly, the variation of the upper jaw resulting from the organic body's feature and the environmental main cause, and its compensation are relatively excluded. The upper and lower jaws are occluded. Accordingly, the variation of the lower jaw can become a variable of the upper and lower jaws. “Position of the teeth when the teeth are clenched is called maximal bite. However, this is not a normal position of a lower jawbone. The normal position is a position when the lower jaw and the upper jaw are widened at a size of 1.5 mm to 5 mm. A space where the lower jaw and the upper jaw are slightly widened therebetween is called “free way” (clinical kinematics published in the Young Mun publisher and written by Seong Su BAE).

13-1. Protraction of Head, and Compensation of Lower Jaw

In case where the head is protracted, if there is not an intentional try to occlude the upper and lower jaw teeth, opening is naturally performed. This can be evaluated that the lower jaw falls and retruded with a standard of the upper jaw. At this time, the lower jaw should elevated and protracted to occlude the upper and lower jaw teeth. For the elevation and protraction of the lower jaw, elevation and protraction muscles of the lower jaw are overstressed. At this time, the lower jaw tooth inclines to push the upper jaw tooth. In case where the lower jaw tooth pushes the upper jaw tooth, a frictional force increases. This helps the lower jaw to resist against the gravity and maintain an bite state. The free way state and a state where the lower jaw tooth pushes the upper jaw tooth are repeated. As a result, the lower jaw inclines to slightly fall and protract for bite. In case where the lower jaw falls, this is a result of reducing the inner pressure upwardly acting against the upper jaw tooth. In case where this is repeated and maintained for a long time, the upper jaw tooth is front and down varied and lingual tilted, and the lower jaw tooth is rear varied and lingual tilted. The down variation of the upper jaw tooth is mainly performed when the free way or the opening is performed. On the horizontal plane, a dentition inclines to be tapered. The dentition front of the lower jaw a little inclines to irregularly slightly overlap or cross. It is inclined for edge-to-edge bite. As a result of front and down varying the upper jaw tooth, the septal cartilage is front and down varied so that the nose inclines to be long and be slightly higher at its top. The lower jaw falls and protracts, and the protraction and elevation muscles of the lower jaw are overstressed.

13-2. Retrusion of Head, and Compensation of Lower Jaw

In case where the head is retruded, the lower jaw inclines for retrusion. This is a main cause of a try to rear vary the weight of the segment to the maximum in correspondence to the retrusion of the head. Hereinafter, it is assumed that the lower jaw is retruded. In case where the lower jaw is retruded, this resultantly reduces the inner pressure acting forwardly to the upper jaw tooth. In case where this is repeated and maintained for a long time, the upper jaw tooth is rear varied and buccal tilted, and the lower jaw tooth is front varied and buccal tilted on the sagittal plane. The forward variation and the buccal tilting of the lower jaw tooth results from the intraoral pressure or the tongue pressure according to case. On the horizontal plane, the dentition inclines to be rectangular. It inclines for deep over bite. In case where the lower jaw is retruded and the dentition is rearranged, or the muscle stress is weakened after the dentition rearrangement, the lower jaw naturally returns to an original position. However, the dentition is rearranged and therefore the state of that time is requested and maintained. At this time, the retrusion muscle or the retrusion and elevation muscles of the lower jaw is or are overstressed. Or, it inclines to bite the tooth. If the tooth is bitten, a frictional force increases. This is helpful for maintaining a retruded state of the lower jaw. Accordingly, a process of retruding and elevating the lower jaw and rearranging the dentition or a state where the free way is observed after the tooth rearrangement, and a state of biting the tooth are repeated. As a result of rear varying the upper jaw, the septal cartilage is rear varied and the nose top inclines to be slightly low. The lower jaw is retruded, and the retrusion and elevation muscles of the lower jaw are overstressed.

13-3. Backward Flexion of Head, and Compensation of Lower Jaw

In case where the head is in the backward flexion, if there is not the intentional try to occlude the upper and lower jaw teeth, the opening is naturally performed. This can be evaluated that the lower jaw falls and retruded with a standard of the upper jaw. At this time, the lower jaw should elevated and protracted to occlude the upper and lower jaw teeth. For the elevation and protraction of the lower jaw, elevation and protraction muscles of the lower jaw are overstressed. At this time, the lower jaw tooth inclines to push the upper jaw tooth. In case where the lower jaw tooth pushes the upper jaw tooth, the frictional force increases. This helps the lower jaw to resist against the gravity and maintain the bite state. The free way state and a state where the lower jaw tooth pushes the upper jaw tooth are repeated. As a result, the lower jaw inclines to slightly fall and protract for bite. In case where the lower jaw falls, this is a result of reducing the inner pressure upwardly acting against the upper jaw tooth. In case where this is repeated and maintained for a long time, the upper jaw tooth is front and down varied and lingual tilted, and the lower jaw tooth is rear varied and lingual tilted. The down variation of the upper jaw tooth is mainly performed when the free way or the opening is performed. On the horizontal plane, a dentition inclines to be tapered. The dentition front of the lower jaw a little inclines to irregularly slightly overlap or cross. There is an inclination for the edge-to-edge bite. As a result of front and down varying the upper jaw tooth, the septal cartilage is front and down varied so that the nose inclines to be long and be slightly higher at its top. The lower jaw falls and protracts, and the protraction and elevation muscles of the lower jaw are overstressed.

13-4. Forward Flexion of Head, and Compensation of Lower Jaw

If the head is in the forward flexion, the lower jaw is naturally protracted and falls by the gravity. The upper and lower jaws are frictionized. In this case, it inclines to avoid the friction of the upper and lower jaw teeth. In order to maintain the free way state free of friction, the retrusion muscle of the lower jaw should be in isometric contraction. However, at this time, the retrusion muscle of the lower jaw is overstressed and the lower jaw inclines to be retruded. Accordingly, the lower jaw is retruded and falls. Hereinafter, it is assumed that the lower jaw is retruded and falls. If the lower jaw is retruded and falls, the upper and lower jaw teeth are distant away. A result of reducing the inner pressure to the upper jaw tooth is obtained. In case where this is repeated and maintained for a long time, the upper jaw tooth is rear and down varied and buccal tilted, and the lower jaw tooth is front varied and buccal tilted. The down variation of the upper jaw tooth is mainly performed when the free way or the opening is performed. The front variation and the buccal tilting of the lower jaw tooth are causes of the intraoral pressure or the pressure of the tongue according to case. On the horizontal plane, the dentition inclines to have an oval shape. There is an inclination for the edge-to-edge bite. In case where the lower jaw is retruded and falls, and the dentition is rearranged, or the muscle stress is weakened after the dentition rearrangement, the lower jaw naturally returns to an original position. However, the dentition is rearranged and therefore the state of that time is requested and maintained. At this time, the retrusion muscle of the lower jaw is overstressed. Or, it inclines to bite the tooth. If the tooth is bitten, the frictional force increases. This is helpful for maintaining a retruded state of the lower jaw. Accordingly, a process where the lower jaw retrudes and falls and the dentition is rearranged or a state where the dentition is rearranged and the free way is observed, and a state of biting the tooth are repeated. As a result of rear and down varying the upper jaw, the septal cartilage is rear and down varied and it is inclined to obtain a Roman nose. The lower jaw is retruded and falls, and the retrusion and elevation muscles of the lower jaw are overstressed.

13-5. Lateral Flexion of Head and Compensation of Lower Jaw

Hereinafter, only a case where the head is buckled will be described. A contrary case can be also understood in the same context. A variation of the lower jaw becomes a variable of the upper jaw tooth. In case where this state is repeated and maintained for a long time, a left upper jaw tooth is left and up varied, and the right upper jaw tooth is left and down varied. In addition, the tongue and the obicularis oris muscle are related to the tooth variation. In case where the head is buckled, the tongue is naturally left varied, and the tooth is pushed to the left. In case where the tongue is habitually pushed to the front, the tooth variation resulting from the tongue increases. An external pressure of a right obicularis oris muscle acting on the upper jaw tooth is increased by the gravity, and an external pressure of a left obicularis oris muscle is decreased. If the gravity naturally has influence, the upper and lower jaw teeth will be all rightly tilted. However, in this case, on the frontal plane, it is inclined that the upper jaw tooth is right tilted but the lower jaw tooth is left tilted. A left tilting of the lower jaw tooth is caused by a little try of the lower jaw intending to maintain an original position resistant against to the gravity (resulting from a balance and esthetic desire) and the tongue acting on the tongue as the variation pressure. Since the lower jaw is left rotated and occluded, it is of being distorted in such as manner that a left and front side of the dentition is protruded, and a right and front side of the dentition is depressed. The septal cartilage is left curved according to the variation of the upper jaw tooth. The head is varied according to the eyesight. At this time, the lower jaw inclines to maintain a possible state of that time. Accordingly, in exercise, the elevation muscle of the lower jaw, and the fall muscle of the right lower jaw are overstressed (At this time, the lower jaw, the variation of the structure related to this, and a variation process can be included). However, the variation is relatively performed within a short time and therefore, this is not considered). For accurate determination, there are many things to be considered and therefore, it is difficult to hastily conclude, but a small exercise repeated and continued for a long time rather than a large exercise performed within a shorter time inclines to be greater reflected on the musculoskeletal system. This is similar even in other segments and particularly is greatly related to formation of various physical figures. This can be understood in the same context that a weak watercourse flowing for a long time perforates a rock.

13-6. Left and Right Rotation of Head, and Compensation of Lower Jaw

Hereinafter, only a case where the head is right rotated will be described. However, a contrary case can be also understood in the same context. In case where the head is right rotated, a right rotation of the lower jaw is less than a right rotation of the upper jaw. This can be evaluated to be of a type where the lower jaw is left rotated with a standard of the upper jaw. Together with this, the head is in the buckling in the right rotation, and is in a right flexion in the left rotation. The variation of the head is similar with the variation described in the “13-5. Lateral flexion of head and compensation of lower jaw”. Accordingly, the variation of the lower jaw cites the variation described in the “13-5. Lateral flexion of head and compensation of lower jaw”. However, in case where there is the same amount of buckling, the variation of the lower jaw and its compensation are larger than those described in the “13-5. Lateral flexion of head and compensation of lower jaw”.

14. Type of Foot

14-1. Flexible Flatfoot

Relatively, a flexible flatfoot results from the weight biased to the external side of the foot. In case where the weight is biased to the external side of the foot, the longitudinal arch reduces. As the front bias of the weight on the foot is accompanied, the longitudinal arch more reduces.

14-2. Rigid Flatfoot

Relatively, a rigid flatfoot results from the weight biased to the front side of the foot. In case where the weight is biased to the front side of the foot, the longitudinal arch reduces. As the external bias of the weight on the foot is accompanied, the longitudinal arch more reduces.

14-3. Pes Cavus

Relatively, a pes cavus results from the weight biased to the rear side of the foot. In case where the weight is biased to the rear side of the foot, the longitudinal arch increases. As the inner bias of the weight on the foot is accompanied, the longitudinal arch more increases.

14-2-4. Hallux Valgus

In an earlier research, it is said that the hallux valgus results from wearing a sharp shoe such as a high-heel. A main cause of the hallux valgus is a sharpness of a shoe portion corresponding to the tiptoe. Actuality is different from this. The hallux valgus is mostly observed when the person whose weight is biased to a front and inner side wears the high-heeled shoe for a long time. In case where the person wears the high-heeled shoe, the weight is biased to the front foot. In posture and exercise, a role of the front foot increases, and a role of the rear foot decreases. At this time, a corresponding role inclines to be biased to the great toe having the strongest muscular strength among the toes. In that case, the weight inclines to be biased at the front and inner side of the foot. In case where this is repeated and maintained for a long time, the weight is biased and built at the front and inner side of the foot and the great toe is curved toward the second toe.

14. Curing

A human posture is naturally formed through a relation of the ground surface and the support structure. In this research, the support structure is provisionally defined as a concept of relatively designating the foot. The human body is finally supported by the foot. A state of the foot supporting the ground surface is greatly related to the human posture. The human is affected by the gravity. In order to effectively erect upright, the foot should firmly support the ground surface, appropriately control the position of the segment, and uniformly distribute the weight of the segment. In case where the weight is biased to a specific position, the balance is broken. For balance, the position of the segment should be again controlled. This is an important cause of the compensation. In case where the posture compensated for the weight bias is performed for a long time, the human body is optimized to the corresponding posture. This includes the variation of the foot. The varied foot requests the continuation of the posture resulting in the corresponding variation. This is an important cause of the formation of the various physical figures. In case where there is a problem in the compensatory mechanism resulting from the weight bias, this problem will be solved by learning and leading so that the weight can be rightly supported. In this research, the variables of the segments are analyzed variously. The analyzed variables of the segments will be used as a useful tool when various human bodies are analyzed and evaluated. In case where there is a problem or a need for curing in the analyzed human body, the variables analyzed in this research can be positively utilized for solution and curing. Solution and curing will be possible by learning a state of the foot, thereby obtaining the solution and the curing effect, and understanding a correlation of the foot state and the posture. At this time, the foot state for obtaining the solution or the curing effect includes all of the ideal state and the state of the foot selected according to need. Here, the state where the weight is supported on the foot is importantly considered. In addition, the variables analyzed in this research can be positively utilized in positively holding a request of the corresponding organic body and solving or improving a problem in a desired state in case where there is a specific state of the human body requested by the corresponding organic body.

The ideal posture in this research is separately defined in sufficient consideration of a feature of an individual organic body and an environmental main cause by the organic bodies. Accordingly, even though it is evaluated to be objectively ideal, in case where it is not determined to be ideal in the corresponding organic body, this can be the object for curing. Further, even though it is evaluated to be objectively negative, in case where it is determined to be ideal in the corresponding organic body, this may not be the object for curing.

15. Remedy

As generally observed, it is inclined that the physical figure reflects the constitution and the constitution reflects the physical figure, thereby co-owning their characteristics on the same or similar extension line. Accordingly, it is possible to understand that the physical figure and the constitution are based on a similar concept even though they are strictly distinguished. In this technological spirit, it is understood that they are similar. The human posture is naturally formed through the relation of the ground surface with the support structure. In case where the posture formed through the ground surface and the support structure is repeated and maintained for a long time, thereby being built, this is defined as the physical figure. The building includes optimization of the musculoskeletal system, and includes all of concepts of reinforcement and weakening of the muscle. In this technological spirit, the variable of the physical figure is analyzed variously. The analyzed variable can be used for curing the physical figure. The physical figure and the constitution are defined as a similar concept. Accordingly, in this research, it is assumed that the curing of the constitution is possible through the curing of the physical figure. Here, the lesion and deformity of the corresponding organic body, and the lesion and deformity resulting from the corresponding physical figure and the constitution are all included. A state of muscle stress relating to posture maintenance shows a different characteristic by types. In a process or its result of forming the physical figure, the stress state of a muscle suppressing an organ can be evaluated to be negative or otherwise states can be evaluated to be positive in the organ. In surgery, in case where excessive impact, suppress, friction and the like results in hurt, bruise, and blister, this is understood as being natural in a common sense of surgery. This should be understood in the same context even in an internal medicine. Accordingly, in case where there is the organ suppressed due to the corresponding posture and physical figure in the internal medicine, this will result in function deterioration or lesion of the corresponding organ, and the deformity. In a contrary case, the corresponding organ can be determined to be positive in function. Accordingly, in case where there is an internal lesion due to the muscle stress suppressing the organ, it can be determined that this is remedied by curing the corresponding posture and physical figure before internal treatment. At this time, this technological spirit can be positively utilized. This technological spirit can be positively utilized not only for the curing of the posture and physical figure but also for the curing and treatment of the constitution, the lesion, and the deformity, and improvement of beauty, learning, and exercise capability. In this case, in case where sufficient learning and understanding for the compensation, and mental treatment are accompanied, a more positive effect can be expected as a result.

### ADVANTAGEOUS EFFECTS

The present invention relates to an apparatus for learning and leading a user so that weight can be supported at feet according to a corresponding purpose, and is characterized in that it is positively utilized for prevention of lesion and deformity, curing and treatment of physical figure, foot, posture, and further constitution and lesion, and deformity, and for beauty and exercise ability improvement, thereby improving a state of a human body.

### BRIEF DESCRIPTION OF THE DRAWINGS

FIG. 1 is top and perspective views schematically illustrating a structure integrated in conformity with a corresponding purpose according to an embodiment of the present invention;

FIGS. 2, 3, 4, and 5 are perspective views schematically illustrating structures manufactured in various types according to embodiments of the present invention;

FIG. 6 is a left-side section view illustrating schematically illustrating a structure having various inserted materials corresponding to a protrusion part and a sinking part when a top plate, a middle plate, and a bottom plate are integrated according to an embodiment of the present invention;

FIG. 7 is a perspective view schematically illustrating a structure having a protrusion part applied to a sock according to an embodiment of the present invention;

FIG. 8 is a perspective view schematically illustrating a structure having a protrusion part applied to a foot according to an embodiment of the present invention;

FIG. 9 is top and left side views schematically illustrating a structure of an electronic curing apparatus according to the present invention;

FIG. 10 is a conceptual view illustrating an operation of an electronic curing apparatus according to the present invention;

FIG. 11 is a conceptual view illustrating a state where a segment position sensor and a muscular strength sensor are applied to a human body according to the present invention; and

FIG. 12 illustrates a pelvis and a foot viewed on a horizontal plane according to the present invention.

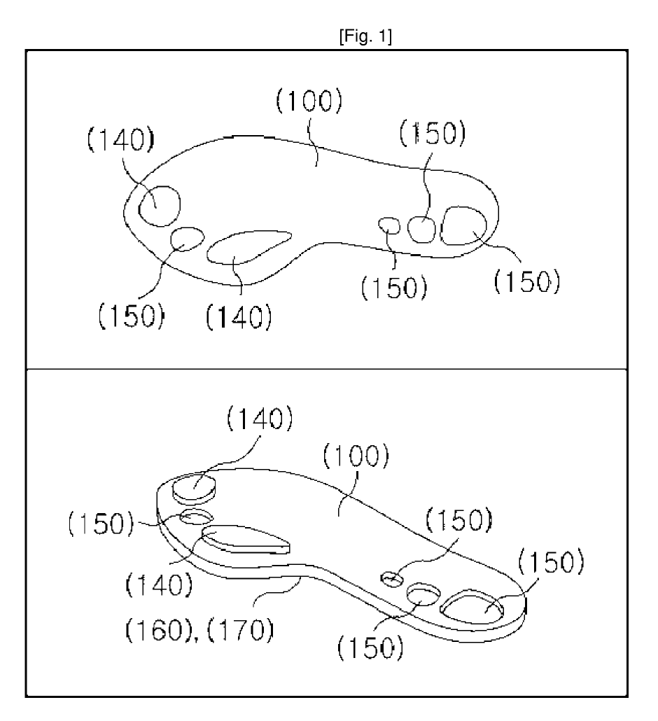

### BEST MODE FOR CARRYING OUT THE INVENTION

Hereinafter, preferred embodiments of the present invention will be described in detail with reference to accompanying drawings.

The present invention can be positioned at and fixed to a foot bottom surface or parts of various objects corresponding the foot bottom surface, or integrated with the corresponding object.

The present invention can be comprised of a slipsole body 100, a protrusion part, a sinking part, a foot print unit 160, a print paper 170, and an electronic curing apparatus. The slipsole body 100 can be constituted of a top plate 110, a middle plate 120, and a bottom plate 130. The foot print unit 160 can be positioned under the slipsole body 100, and the print paper 170 can be positioned under the foot print unit 160.

A structure according to the present invention can be varied according to need in position, type, size, material, structure, and construction. The structure can be partially different in type, size, material, structure, and construction, respectively, and can be constructed through a combination of a plurality of fragments.

The structure corresponding to the foot bottom surface can constitute one set by two, and can be applied to each of both feet and only to a specific foot according to need.

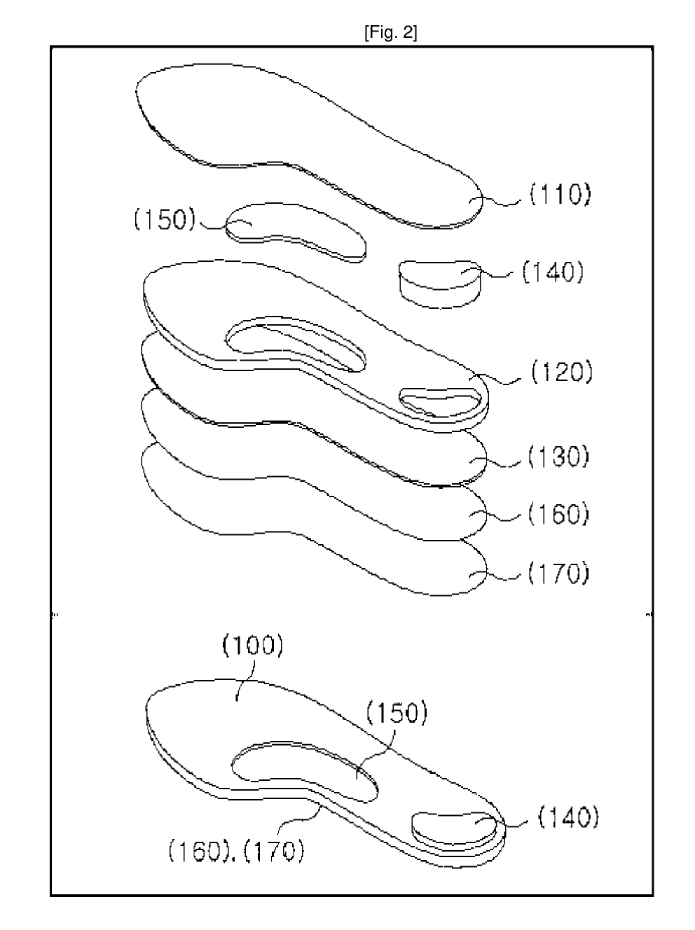

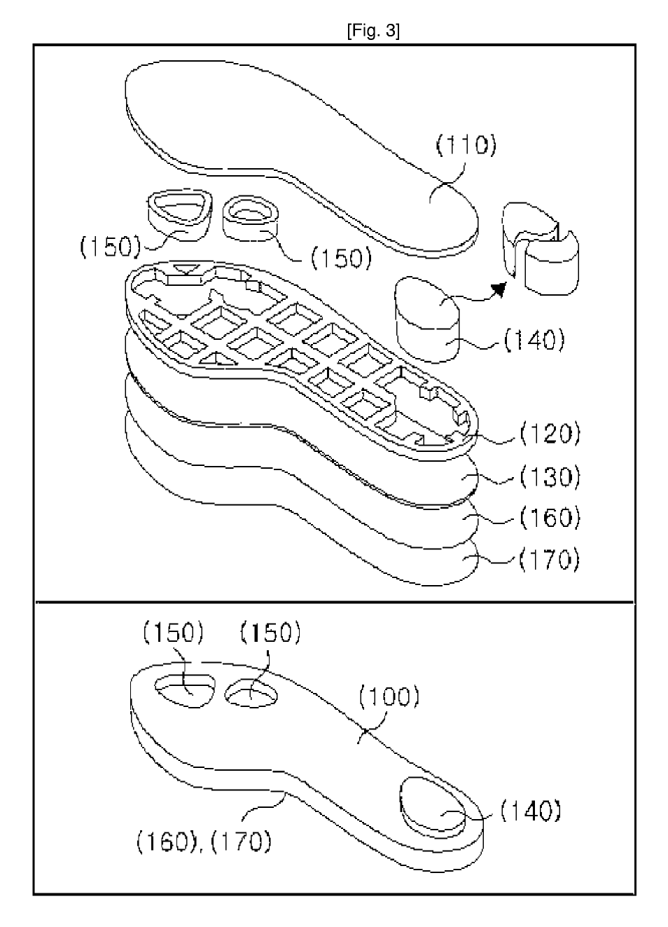

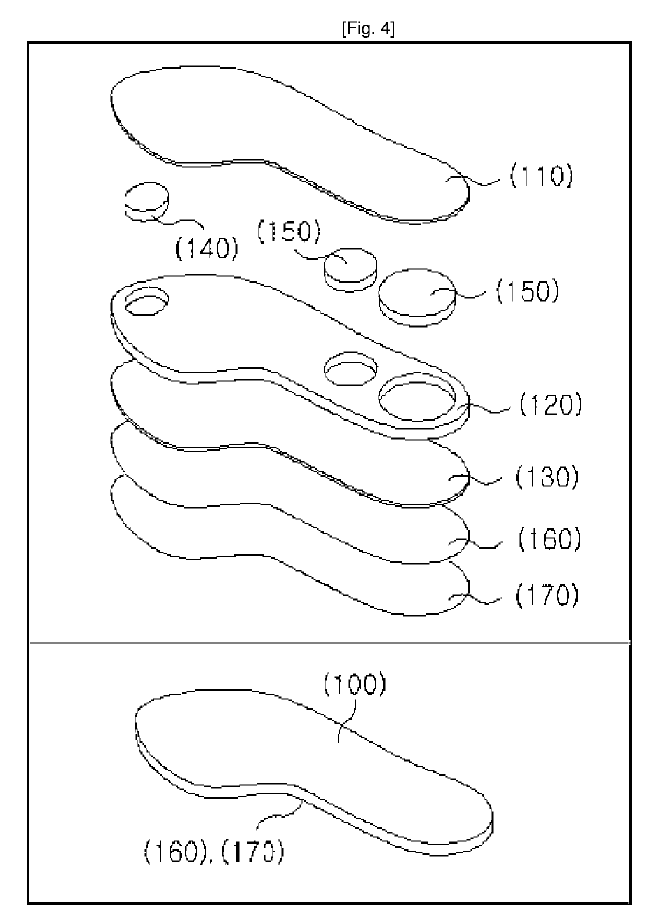

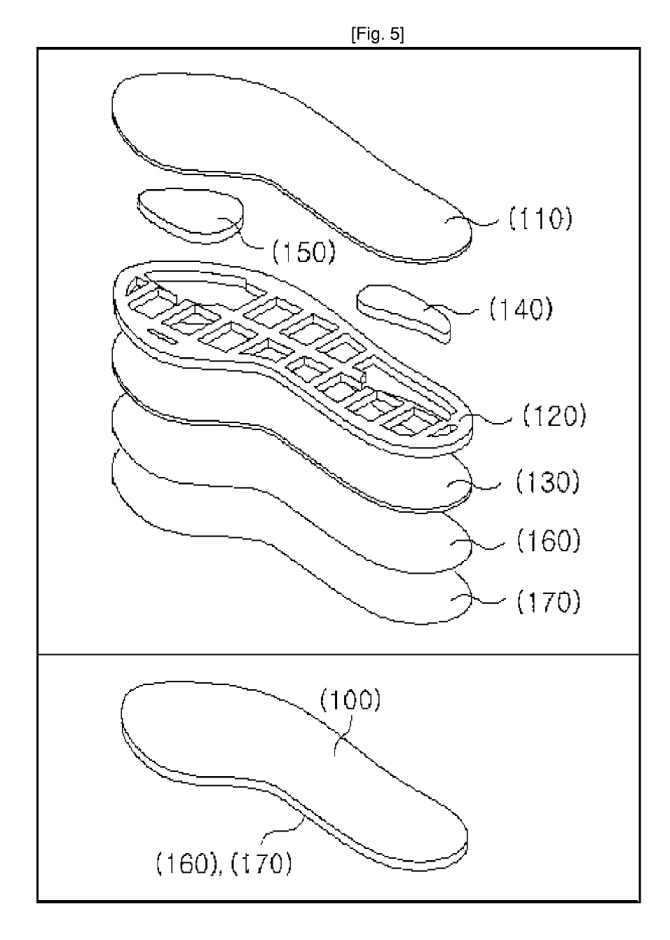

FIGS. 2, 3, 4, and 5 schematically illustrate examples of the structures manufactured in various methods and constructions.

The inventive structure can be omitted in its partial construction, and reconstructed in conformity with the corresponding purpose according to need. At this time, a spirit of the present invention, and a feature and a function of a corresponding structure are identically applied.

The protrusion part and the sinking part are positioned at the foot for supporting a user's weight, and allow a user to recognize the corresponding purpose such as a degree of weight loaded on the foot, using a sense of the foot bottom surface, and are positioned at the slipsole body 100 and learning and leading the user to support his/her weight on the feet in conformity with the corresponding purpose.

The protrusion part and the sinking part can be manufactured and functioned on the basis of various principles, structures, and constructions. A protruded portion can be formed on the slipsole body 100 to function as the protrusion part, and a sinking portion can be formed on the slipsole body 100 to function as the sinking part. In the structure functioning as the protrusion part and the sinking part, it is manufactured to be of a type not protruded or sunken on an upper surface of the slipsole body 100 when the weight is not loaded. However, a structure less pressed in comparison with an upper surface of the slipsole body 100 can function as the protrusion part, and a more pressed structure can function as the sinking part. According to need of the corresponding purpose, the methods can be combined for manufacture.

When the weight is loaded, the structure functioning as the protrusion part can be pressed by the weight, thereby being or not in the same as or similar height with the upper surface of the slipsole body 100. When the weight is loaded, thereby pressing the upper surface of the slipsole body 100, the structure functioning as the sinking part can have or cannot have the same as or similar height with the upper surface of the slipsole body 100.

A protruding unit 140 has a structure being capable of functioning as the protrusion part, and a sinking unit 150 has a structure being capable of functioning as the sinking part. The protruding unit 140 and the sinking unit 150 are inserted into a hole of the middle plate 120. The protruding unit 140 and the sinking unit 150 can have size, type, and construction corresponding to or similar with those of the hole of the middle plate 120 when viewing on their top views.

The protruding unit 140 can be manufactured to be higher than the hole of the middle plate 120, thereby consequently forming the protruded portion on the upper surface of the slipsole body 100 and functioning as the protrusion part. The protruding unit 140 can be manufactured to have the same height as the hole of the middle plate 120 but formed of material having a greater elasticity than the middle plate 120 so that, when the weight is loaded, it can be consequently less pressed than other portions of the slipsole body 100 or not pressed, thereby functioning as the protrusion part. According to need, the methods can be combined for manufacture. For the purpose of assisting a function of the protrusion part, the protruding unit 140 can be combined with a spring on its upper and lower surfaces.

The sinking unit 150 can be manufactured to be lower than the hole of the middle plate 120, thereby consequently forming the sunken portion on the upper surface of the slipsole body 100 and functioning as the sinking part. The sinking unit 140 can be manufactured to have the same height as the hole of the middle plate 120 but formed of material having a smaller elasticity than the middle plate 120 so that, when the weight is loaded, it can be consequently more pressed than other portions of the slipsole body 100, thereby functioning as the sinking part. According to need, the methods can be combined for manufacture. In addition, according to case, the sinking unit 150 can be formed of the same material as the middle plate 120, with a corresponding or similar size with the hole of the middle plate 120, and be inserted into the middle plate 120, thereby functioning as a part of the middle plate 120.

All or any one of the protruding unit 140 and the sinking unit 150 can be applied or not applied to the hole of the middle plate 120 according to the corresponding purpose. In case where the protruding unit 140 and the sinking unit 150 are all not applied to the hole of the middle plate 120, the hole of the middle plate 120 can be functioned as the sinking part.

The protruding unit 140 and the sinking unit 150 can have a structure based on a principle of a flute, or a structure being capable of sounding by an operation of an electronic device according to need.

The structure functioning as the protruding part and the sinking part can vary in position and number according to need, and can be differently constructed in portions corresponding to both feet, respectively. The protruding part and the sinking part are positioned at the slipsole body 100 according to the corresponding purpose and therefore, may not be consistent with an ideal position of the user's foot at which the weight should be supported.

In case where it is judged that the user is easy to recognize the corresponding purpose with a standard of the protrusion part and effectively achieves the corresponding purpose in applying the present invention according to the corresponding purpose, the protrusion part can be provided on the upper surface of the slipsole body 100 according to the corresponding purpose and recognized in conformity with the corresponding purpose so that the corresponding purpose can be learned and lead. At this time, the sinking part may not be provided.

On the contrary, in case where it is judged that the user is easy to recognize the corresponding purpose with a standard of the sinking part and effectively achieves the corresponding purpose in applying the present invention according to the corresponding purpose, the sinking part can be provided on the upper surface of the slipsole body 100 according to the corresponding purpose and recognized in conformity with the corresponding purpose so that the corresponding purpose can be learned and lead. At this time, the protrusion part may not be provided.

Further, in case where it is judged that the user is easy to recognize the corresponding purpose with a standard of a combination type of the protrusion part and the sinking part and effectively achieves the corresponding purpose in applying the present invention according to the corresponding purpose, the protrusion part and the sinking part can be provided in a combination type on the upper surface of the slipsole body 100 according to the corresponding purpose and recognized in conformity with the corresponding purpose so that the corresponding purpose can be learned and lead.

In addition, in case where it is judged that the user is easy to recognize the corresponding purpose with a standard of a portion of the slipsole body 100 excepting the protrusion part and the sinking part and effectively achieves the corresponding purpose in applying the present invention according to the corresponding purpose, the protrusion part and the sinking part can be provided on the upper surface of the slipsole body 100 according to the corresponding purpose and recognized in conformity with the corresponding purpose so that the corresponding purpose can be recognized with a standard of the portion of the slipsole body 100 excepting the protrusion part and the sinking part, thereby allowing the corresponding purpose to be learned and lead. At this time, all or any one of the protrusion part and the sinking part can be provided according to need.

The slipsole body 100 is manufactured to have a size corresponding to all or any part of the foot bottom surface, and function as a slipsole base. The slipsole body 100 is a body of a slipsole being capable of including the protruding unit 140 and the sinking unit 150.

In the present invention, in case where the user does not clearly recognize the protrusion part and the sinking part, which are formed according to the corresponding purpose or for the purpose of accurately supporting the weight according to the corresponding purpose, or needs a help, the upper surface of the slipsole body 100 being in contact with the foot bottom surface can be varied in plane height or in application in consideration of a state of the corresponding user's foot for the purpose of helping. In that case, the structure included in the slipsole body 100 can be adapted suitably to a state of the slipsole body 100 within a range conforming with the corresponding purpose.

In application, in case where the user has both legs different in length, for the purpose of accurately recognizing the protrusion part and the sinking part according to the corresponding purpose, the slipsole body 100 and the corresponding structure applied to the slipsole body 100 can be each varied in length in the structure corresponding to both feet. The middle plate 120 can have a plurality of holes varying in position, number, type, size, and construction according to need. The top plate 110 and the bottom plate 130 can act as a role of fixing the protruding unit 140 and the sinking unit 150 applied to the middle plate 120. The top plate 110 is connected to the upper surface of the middle plate 120, and the bottom plate 130 is connected to the lower surface of the middle plate 120. In case where the top plate 110 and the bottom plate 130 are consequently connected to the middle plate 120 having the protruding unit 140 and the sinking unit 150 applied thereto, they can have a type adapted to a state of the middle plate 120 including the protruding unit 140 and the sinking unit 150. The connected top plate 110, middle plate 120, and bottom plate 130 can have the same or similar size when viewing on its top view (view from the upper side). The top plate 110 does not function as the protrusion part, but can have a predetermined prominence and depression for the purpose of accurately recognizing the corresponding purpose or the purpose of anti-slip, acupressure, and reflex zone therapy.

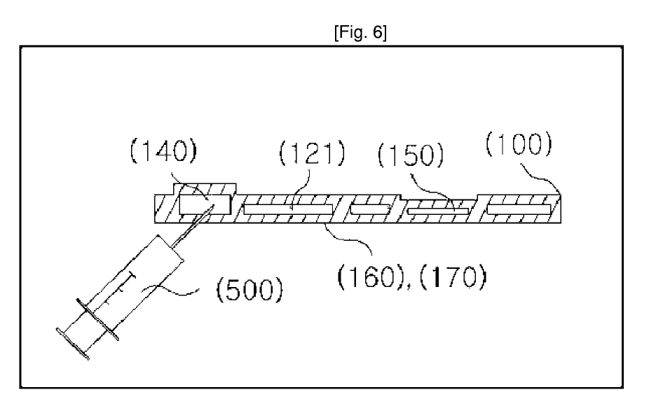

The top plate 110 and middle plate 120, the middle plate 120 and bottom plate 130, or the top plate 110, middle plate 120 and bottom plate 130 can be at first manufactured in an integrated type. In case where the top plate 110, the middle plate 120, and the bottom plate 130 are manufactured in the integrated type, a necessary amount of various materials such as air, liquid, silicon, rubber, and synthetic resin is inserted into a portion 121 corresponding to the hole of the middle plate 120 using an injector or a suitable method according to need, and is processed to maintain in type, to be served as the protrusion part and the sinking part (FIG. 6).

The inventive structure can be manufactured using various materials having a suitable physical property, and can be manufactured using various methods such as a known shaping technology corresponding to a suitable method and its applied method. The present invention can be at first integrated and manufactured, and can be integrated and manufactured using a structure having a corresponding property or combining its fragments. This can be easily applied by those skilled in the art in its manufacture process.

In the inventive structure, various methods can be used for the purpose of connection and fixation, such as a method of providing a groove to a structure such as adhesive, screw, bolt, and nut having a suitable physical property, and its part, providing a protrusion portion corresponding to the groove to its corresponding structure, and fitting the protrusion portion into the groove.

In application, the present invention can be manufactured as an independent typed slipsole, and be applied to an inner lower surface of the shoe, and be functioned as a slipsole, and can be connected to a lower surface of the shoe corresponding to the foot bottom surface and integrated with the shoe. In addition, for a person doing many indoor activities, the present invention can be manufactured in an independent type and inserted into the sock 200, or can be integrated with the sock 200 according to the corresponding purpose.

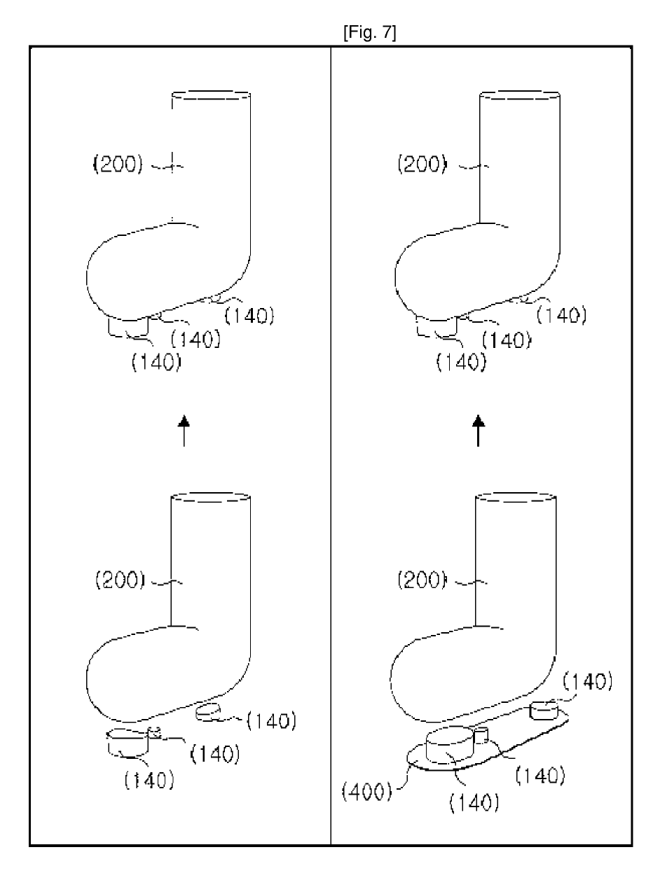

In the present invention, the partial structure can be omitted according to the corresponding purpose. In application to the sock 200, only the protruding unit 140 can be manufactured according to the corresponding purpose, and can be connected to an inner side or an outer side of the sock 200 corresponding to the foot bottom surface according to the corresponding purpose (FIG. 7).

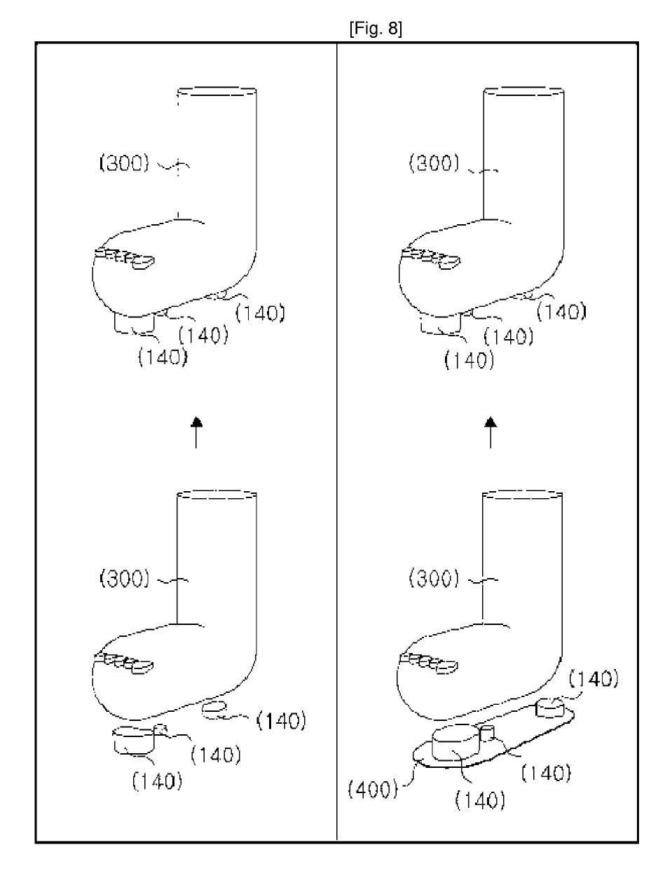

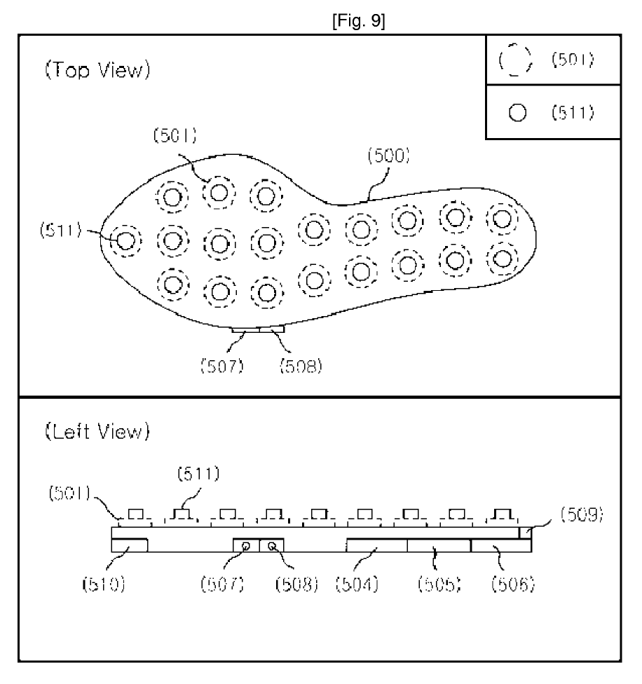

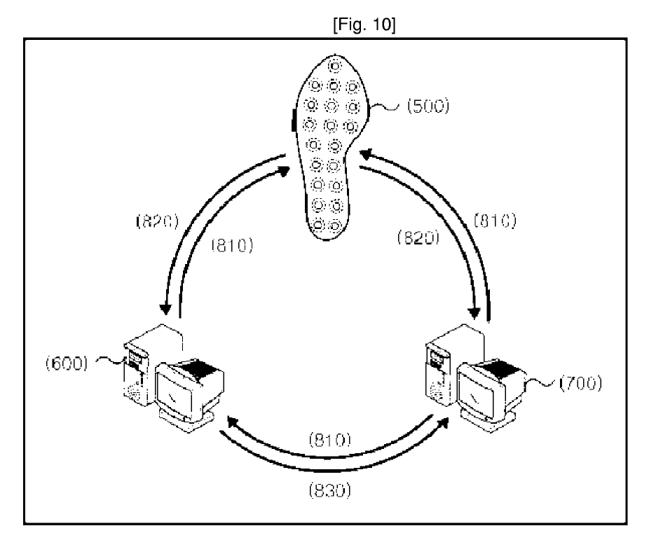

In addition, in application to the sock 200, the user can directly apply the protruding unit 140 to the sock 200 according to the corresponding purpose (FIG. 7). At this time, for convenience, the bottom plate 130 is used as a transfer paper 400 having a size and a type corresponding to the foot bottom surface, and the protruding unit 140 can be attached to the transfer paper 400 according to the corresponding purpose. In this case, the user attaches the protruding unit 140 to the sock 200 using the structure, and removes the transfer paper 400. In attachment, a suitable adhesive can be used. In addition, the protruding unit 140 can be manufactured using material having an adhesive force when heat is applied, so that the protruding unit 140 is heated and attached to the sock 200 (FIG. 7). This is similar with a method where the transfer paper having a pattern and a letter is put on and pressed on clothes, thereby attaching and printing the corresponding pattern and letter to the clothes. In case where the adhesive is used, it can be directly applied to the foot 300, not the sock 200 (FIG. 8). In this case, an easily detachable adhesive can be used. In application to the foot, the corresponding structure can be constructed and fixed to the foot according to the need. In this case, an apparatus such as a string and a belt can be used. In that case, a structure such as the string and the belt can be fixed to the corresponding structure.

The foot print unit 160 can have a carbon paper or material having a similar function, and can have a size corresponding to the lower surface of the slipsole body 100. The foot print unit 160 imprints the footprint, and imprints a degree of a locally loaded weight on the underlying print paper 170 with color.

The print paper 170 is manufactured using various materials allowing the functions, and connected or positioned to allow attachment on and detachment from the lower surface of the foot print unit 160. The print paper 170 can have a corresponding or similar size with the foot print unit 160.

In case where the electronic curing apparatus 500 is applied, the foot print unit 160 and the print paper 170 can be omitted.

The electronic curing apparatus 500 is an apparatus for operating based on a curing program 810, and allowing the learning of the curing program, and can wire or wireless communicate and share data and operate with a terminal device 600 and a Web site 700.

In the electronic curing apparatus 500, its partial structure can be externally positioned and fixed in an independent type to wire or wireless communicate and share data and operate with a corresponding device.

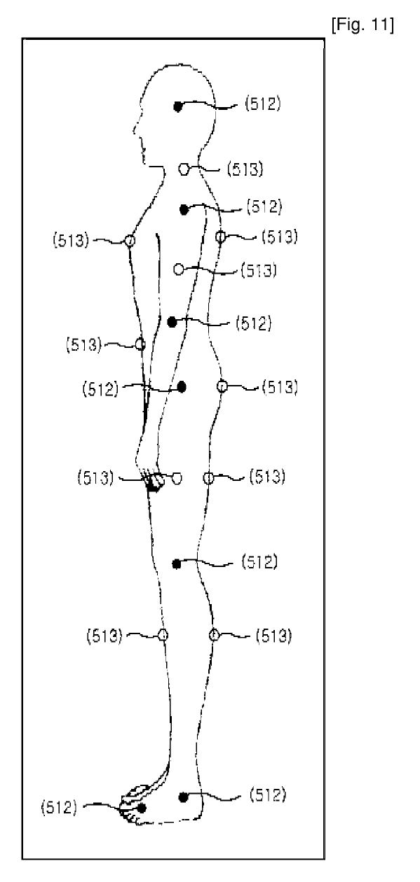

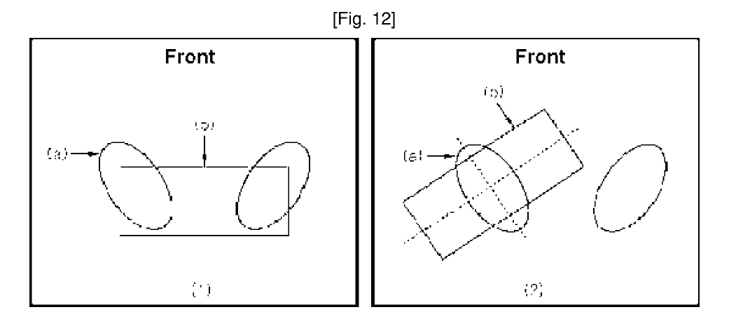

The electronic curing apparatus 500 can operate based on the curing program, and include a management system; an Internet protocol unit; a device such as a wire or wireless communication modem and a network adapter; a foot bottom pressure sensor 511 (it can be constructed in plural. In case where it is constructed in single, it can have a size corresponding to the corresponding foot) for sensing a degree of a locally loaded weight and outputting the sensed degree to a curing program master 504; the segment position sensor 512 attached to the segment of the human body according to need and sensing a position of the corresponding segment to wire or wireless output the sensed position to the curing program master 504; the muscular strength sensor 513 (FIG. 11) for sensing and wire or wireless outputting a corresponding muscular strength to the curing program master 504; the curing program master 504 for analyzing and processing data received from the foot bottom pressure 511, comparing and analyzing the data with the curing program 810, operating each device based on the curing program 810, and outputting corresponding data to each device; a database 506 for storing the data, which is received from the foot bottom pressure sensor 511 and analyzed and processed in the curing program master 504, (Hereinafter, referred to as “learning data”, and the learning data including a statistic for a position, a degree, and a time where the weight is loaded, by parts, degrees, and times) and the curing program 810; a database managing unit for requesting the database 506 to input and output the data; and an output unit 508 for wire or wireless outputting the learning data 820 and the curing program 810 to the terminal device 600 and the Web site 700; an input unit 507 for wire or wireless receiving the curing program 810 from the terminal device 600 and the Web site 700; a signaling unit (as various units being capable of performing the above function, there are a video part such as a monitor and a liquid crystal screen; a sound part for outputting a corresponding message using a voice message and a signal sound; and a vibration unit for outputting the corresponding message using vibration) for informing the user of a corresponding content using video data, voice message, signal sound, and vibration when there is an error message in position and degree where the weight should be loaded, position and degree where the weight should not be loaded, and operation of the electronic curing apparatus 500, and a message of upgrading the curing program 810; a control unit for checking the message and manipulating an operation corresponding to the message such as execution and cancellation (the control unit can include a check unit for, when there is a signal requesting a user check, checking the user and commanding discontinuance of the corresponding signal, an execution unit for commanding execution of the curing program, and a cancellation unit for commanding cancellation of the execution); and a battery 510 for operating each device, and a socket being connectable with an adapter.

In case where the electronic curing apparatus 500 has the segment position sensor 512 and the muscular strength sensor 513, it can be associated with the segment position sensor 512 and the muscular sensor 513, and the curing program master 504 can analyze and process the corresponding data sensed in the segment position sensor 512 and the muscular sensor 513. In this case, the curing program can have the corresponding data necessary for the operation of the segment position sensor 512 and the muscular sensor 513, and can have the signaling unit that is positioned on the basis of the curing program, generates a signal for exercise, and informs the user.

The weight being biased to the foot causes the variation of the segment. The electronic curing apparatus is an apparatus for diagnosing and curing a support state of the weight on the foot. However, in case where the foot has irregular variation, the electronic curing apparatus can erroneously operate in diagnosing a state of the human body at that time. In case where the segment position sensor 512 and the muscular sensor 513 is associated with the foot bottom pressure sensor 511, a state of loading the weight on the foot and a state of the segment and the muscular strength at that time are synthesized and the corresponding human body is analyzed and evaluated, thereby reducing a possibility of error, and accurately diagnosing the state of the user's body.

The terminal device 600 corresponds to various devices being capable of performing the above and below operations such as a personal computer (PC), portable digital assistants (PDA), and a cellular phone. The terminal device 600 can be wire or wireless connected and operated with the electronic curing apparatus 500 and the Web site 700.

The terminal device 600 can include a management system; a device such as a wire or wireless communication modem and a network adapter; an Internet protocol unit; Browser; an output unit for wire or wireless outputting the curing program 810 to the electronic curing apparatus 500; an input unit for wire or wireless receiving the learning data 820 and the curing program 810 from the electronic curing apparatus 500; a database management unit for requesting the database to input and output the data; an output unit such as a monitor and a liquid crystal screen; an input device such as a key board, a mouse, and a touch pad; and a curing program manager being capable of editing and modifying the curing program 810 (it can receive the learning data and the curing program from the electronic curing apparatus and the Web site. The received learning data 820 can be used for edition, modification and statistic of the curing program 810 in the future.

The Web site 700 has a server device. The server device can be connected with the electronic curing apparatus 500 and the terminal device 600 through Internet, and communicate the learning data 820 and the curing program 810.

The server device can include a management system; a device such as a wire or wireless communication modem and a network adapter; an output unit such as a monitor and a liquid crystal screen; an input unit such as a keyboard, a mouse, a touch pad; an Internet protocol unit; a WWW server; a CGI program unit for providing the curing program 810, and analyzing and processing the learning data; a database; and a database management unit for requesting the database to input and output the data.

In operation of the electronic curing apparatus 500, the curing program master 504 can be programmed to periodically search a Web site database and automatically download a new curing program 810 in case where the electronic curing apparatus 500 receives the curing program 810 from the Web site 700. When the new curing program is downloaded, the curing program master 504 commands the signaling unit to generate the corresponding signal (Step 100). If the signal is checked and the check unit 509 is manipulated, the signal is discontinued. If the execution unit is manipulated in the Step 100 (Step 200), execution of the curing program 810 is discontinued, and storage of the new curing program 810 is requested and stored in the database management unit (Step 300). In case where a storage space is deficient in the Step 200, the corresponding message is informed through the signaling unit (Step 400). In case where the execution unit is manipulated, the long-standing curing program 810 is deleted as required, and the new curing program 810 is stored (Step 401). In case where the cancellation unit is manipulated in the Step 400, the storage is canceled and the given curing program 810 is again executed. After the progressing of the Steps 300 and 401, the curing program master 504 allows the device to operate according to the new curing program 810.

The foot bottom pressure sensor 511 can be different in number and position according to need, and can be positioned and fixed to the foot bottom surface such as the foot, the slipsole and the sock or to a surface of a variety of objects corresponding to the foot bottom surface to sense the degree where the weight is loaded by parts, on the basis of the curing program 810. The data sensed by the foot bottom pressure sensor 511 is inputted to the curing program master 504. The curing program master 504 operates the device based on the curing program 810, analyzes and processes the received data, compares the data with the curing program 810 for analysis and processes in a format of the learning data 820, requests and stores the learning data 820 in the database management unit, and outputs the data to the terminal device 600 and the Web site 700 wire or wireless connected through the output unit 508. The terminal device 600 and the Web site 700 store the learning data 820 in the database of the corresponding device.

The curing program 810 can be stored in the database 506 such as the electronic curing apparatus 500, the terminal device, and the Web site 700. The curing program 810 has the degree by parts where the weight should be loaded on the foot, the position and degree where the weight should not be loaded, the position of the corresponding segment, the corresponding muscular strength, and the operation content of each device, and follows the corresponding purpose. It is desirable that the curing program 810 is different in content by learning steps and progress rates. The learning data can be checked, modified, deleted using the curing program manager, and the curing program 810 can be manufactured, modified, deleted, and downloaded from the Web site 700. The curing program 810 can be inputted, stored, and executed in the electronic curing apparatus 500.

The operation of the curing program manager requests the database management unit of each device for the learning data 820 and the curing program 810. The database management unit searches the database, and outputs the searched data to the curing program manager. The curing program 810 is manufactured, modified, stored, and outputted to the electronic curing apparatus 500 (Step M100). The curing program master 504 discontinues the execution of the given curing program 810, and requests and stores the curing program 810 in the database management unit, and executes the new curing program 810.

In case where the curing program 810 is modified through the Web site 700, connection is made to the Web site 700 through the browser of the terminal device, and modification of the curing program 810 is requested (Step R100). The CGI program unit edits a curing program modification page with a standard of the database of the server device, and outputs the edited program to the WWW server. The WWW server outputs the edited program to the browser of the terminal device. The user modifies the curing program, and transmits the modified curing program to the Web site 700. The WWW server outputs the received data to the CGI program unit. The CGI program unit analyzes and processes the data, and edits the curing program with a standard of the database, and requests and stores the curing program in the database management unit (At this time, the curing program can be outputted to the electronic curing apparatus). The CGI program unit edits the storage result and the modified curing program into a curing program modification completion page, and outputs the edited completion page to the WWW server. The WWW server unit outputs the completion page to the browser of the terminal device. The check by the user is performed (Step C100), and the modified curing program is requested and stored in the database management unit of the terminal device. Steps subsequent to the Step M100 can be performed.

In case where the learning is accurately performed or not, the signaling unit can generate the signal so that the user can recognize the corresponding content. In case where the segment position sensor is provided, when the corresponding segment is positioned or not at its essential position, the corresponding signal can be generated and the user can be informed according to the curing program 810 (in case where exercise is performed over a long time, and in case where an exercise pattern is analyzed and does not follow the curing program, the user can be informed using the signal. In the same manner as the operation of the segment position sensor in case where the muscular strength sensor is provided, the operation can be performed.

The signaling unit is controlled in operation by the curing program 810. The control content can be varied through the modification of the curing program 810. For one example, as long as the learning is normally performed or not more than predetermined times with accumulation points placed, the signal can be generated.

In case where the weight is not loaded on the electronic curing apparatus 500 according to the curing program 810, the curing program master 504 generates a signal through the signaling unit, and informs the user.

In case where the electronic curing apparatus 500 is associated with the corresponding device such as the Web site wire or wireless providing the curing program 810, it can output the learning data 820 to and associate with the corresponding device in real time. In that case, the corresponding device analyzes, evaluates and processes the received learning data 820, and modifies the progress rate-based curing program 810 when the learning is accurately performed according to the curing program 810 by progress rates, and modifies and outputs its corresponding curing program 810 to the electronic curing apparatus 600 when the learning is not accurately performed.

The terminal device 600 and the electronic curing apparatus 500 can have an integrated type. In that case, a duplicate structure can be united and an unnecessary structure can be omitted. For one example, the electronic curing apparatus can be manufactured on its lower surface to have a type of the liquid crystal screen and the keyboard.

The electronic curing apparatus 500 can be manufactured in the independent structure and combined with other structures of the present invention, and can be embedded in the protruding unit 140 and the sinking unit 150 to perform the corresponding function. For one example, in case where the protruding unit 140 is singly applied to the protruding unit 140 such as the sock and the foot, the electronic curing apparatus 500 can be embedded in the corresponding protruding unit 140.

In an applied example of the present invention, the corresponding structure employs the same spirit and property of the structure of the present invention.

The present invention is manufactured according to the corresponding purpose and applied to the corresponding user. However, in case of the users showing similar types, the similar types can be classified in one type, and a model commonly applicable to the classified type can be mass-produced and collectively applied. The model can be mass-produced in a format of allowing partial modification by types and partially modified according to the corresponding purpose and individually applicable to the corresponding user.

### INDUSTRIAL APPLICABILITY

The present invention provides an apparatus for learning and leading a user to have a posture according to a corresponding purpose at his/her foot, and can cure and improve a state of a human body according to the corresponding purpose.

While the present invention has been described and illustrated herein with reference to the preferred embodiments thereof, it will be apparent to those skilled in the art that various modifications and variations can be made therein without departing from the spirit and scope of the invention. Thus, it is intended that the present invention covers the modifications and variations of this invention that come within the scope of the appended claims and their equivalents.

1. An apparatus for curing a human body using a pressure under foot, wherein a protrusion part and a sinking part are provided under the foot.

2. The apparatus according to claim 1, wherein the apparatus is a slipsole, wherein the slipsole comprises a top plate, a middle plate provided at a lower side of the top plate and having at least one hole, a bottom plate provided at a lower side of the middle plate, and an insertion member inserted into the hole of the middle plate, and wherein the protrusion part or the sinking part is formed by a difference between the insertion member and the middle plate.

3. The apparatus according to claim 2, wherein the protrusion part or the sinking part is formed by a difference in height between the insertion member and the middle plate.

4. The apparatus according to claim 2, wherein the protrusion part or the sinking part is formed by a difference in elasticity between the insertion member and the middle plate.

5. The apparatus according to claim 2, further comprising: a foot print unit provided at a lower side of the bottom plate and having a size corresponding to a lower surface of the bottom plate, and a print paper provided at a lower side of the foot print unit and having a size corresponding to the foot print unit, wherein the foot print unit imprints a footprint on the print paper according to the pressure under foot.

6. The apparatus according to claim 5, wherein the foot print unit is a carbon paper, and the print paper is a paper.

7. The apparatus according to claim 1, further comprising an electronic curing device, wherein the electronic curing device comprises:
a foot bottom pressure sensor positioned on a foot bottom surface or a lower surface of an object corresponding to the foot bottom surface, and sensing a pressure by parts where weight is loaded;
a signaling unit for generating a signal; and
a curing program master for receiving a curing program having information on a position and a degree by parts where the weight should be loaded and its corresponding information on an operation content of the signaling unit stored in a database, and receiving data sensed by the foot bottom pressure sensor, and comparing and analyzing the received information and the information of the curing program, and controlling the signaling unit according to the information on the operation content of the signaling unit included in the curing program.

8. The apparatus according to claim 7, wherein the electronic curing apparatus can be wire or wireless connected with the terminal device or the Web sit, and can modify the curing program through the terminal device or the Web site.

9. The apparatus according to claim 7, wherein the electronic curing device further comprises a segment position sensor for sensing a segment position and a muscular strength sensor for evaluating a muscular strength of a muscle, and the curing program further comprises information on the segment position and the muscular strength for a corresponding posture.

10. A method for curing a human body, wherein a user can be learned and educated for a position at which weight can be supported on feet, thereby curing and improving lesion, prevention of deformity, and the human body.

11. The apparatus according to claim 2, wherein a structure with protrusion part and sinking part makes sound corresponding to weight loaded.

12. The apparatus according to claim 1, wherein protrusion is applied to outer or inner side of sock adjacent to foot bottom.

13. The apparatus according to claim 2, wherein bottom plate is made to the size of foot bottom surface and the protrusion on the bottom plate is provided with adhere so that the protrusion can be attached to a sock or foot bottom surface.

14. The apparatus according to claim 2, wherein the slipsole is positioned at and fixed to parts of various objects corresponding the foot bottom surface, or integrated with the corresponding object.
